(19) United States
(12) Patent Application Publication
(10) Pub. No.: US 2014/0316204 A1
(43) Pub. Date: Oct. 23, 2014

(54) Removably Attachable Endoscopic Water Bottle Assembly and A Flushing Adaptor Thereof

(71) Applicant: EndoChoice Inc., Alpharetta, GA (US)

(72) Inventors: Yaniv Ofir, Givat Haim Ihud (IL); Golan Salman, Atlit (IL); Amram Aizenfeld, Ramot Menashe (IL)

(73) Assignee: EndoChoice Inc., Alpharetta, GA (US)

(21) Appl. No.: 14/228,254

(22) Filed: Mar. 28, 2014

Related U.S. Application Data
(60) Provisional application No. 61/806,043, filed on Mar. 28, 2013.
(60) Provisional application No. 61/881,427, filed on Sep. 23, 2013.

(51) Int. Cl. A61B 1/12

(52) U.S. Cl. CPC A61B 1/121
USPC 600/158

(57) ABSTRACT

The present specification describes an endoscopic water bottle assembly including a cap configured to attach a water bottle and having a port configured to receive a supply tube; a supply tube assembly, having a distal end and a proximal end; and a water bottle configured to receive said cap, wherein the cap includes a first coupling portion to removably attach the water bottle to a second coupling portion, which is attached to an endoscopic main control unit and is configured to receive said first portion of the coupling system. The present specification also describes a flushing adaptor for connecting a fluid source to a dual lumen supply tube, where the flushing adaptor includes a first opening, a second opening, and a housing with a chamber for fluidly connecting said first and second openings, that enables fluid received from said first opening to be distributed throughout the tube via the connector.

### CROSS REFERENCE

The present specification relies on U.S. Provisional Patent Application No. 61/806,043, entitled “A Removably Attachable Endoscopic Water Bottle Assembly”, and filed on Mar. 28, 2013 for priority. In addition, the present specification relies on U.S. Provisional Patent Application No. 61/881,427, entitled “Flushing Adaptor for An Endoscopic Water Bottle”, and filed on Sep. 23, 2013, for priority.

All of the above-mentioned applications are herein incorporated by reference in their entirety.

### FIELD

The present specification generally relates to endoscopic systems and more particularly to advantageous positioning/placement of a reusable endoscopic water bottle assembly with reference to the operating environment of the endoscopic systems and to a flushing adaptor that enables cleaning fluid to be uniformly distributed through the lumens of a concentric dual lumen supply tube of the reusable endoscopic water bottle.

### BACKGROUND

Endoscope systems typically include a plurality of sub-systems, ancillary equipment, components, connectors and tooling. For example, an endoscopy system comprises an elongated flexible cable equipped at one end with an eyepiece or other viewing mechanism, such as a main control unit that displays a video of the internal anatomical details as the tip of the endoscope is advanced into a human body. Further, the endoscope includes inlets/connectors that couple long supply tubes that enable fluids, such as water and/or gas, to flow into the endoscope. These supply tubes still further include inlets/connectors to couple them to water and/or gas sources such as an endoscopic water bottle and/or a CO₂ insufflator.

An endoscopic operating environment is therefore fairly cluttered with associated tubes, power cables, connector knobs, fluid sources, medical tools, etc. In such an environment, the endoscopic water bottle typically stands independently and requires a nurse or caregiver to hold or track the bottle so that it does not fall, get caught up or entangled in other tubing.

Also, an endoscope system that utilizes a reusable water bottle for supplying irrigation fluid during endoscopic operations needs to be cleaned and disinfected between uses. One of the ways of cleaning the water bottle as well as its dual lumen tube (that supplies irrigation fluid to an endoscope) is to purge both with a cleaning fluid, such as water, under pressure.

There is thus a need in the art for a reusable endoscopic water bottle assembly that is easy to manipulate and can be held securely while unattended such that it does not fall or get tangled during endoscopic operations. There is also a need in the art to place and/or position the reusable endoscopic water bottle assembly in a manner that is removably, yet securely, attachable within the endoscopic operating environment.

There is also a need for a system that enables efficient and effective flush cleaning or purging of a dual lumen tube of the reusable endoscopic water bottle. It is desirable to have a flushing adaptor that enables streamlined and uniform distribution of cleaning fluid through the two lumens of the dual lumen tube.

### SUMMARY

The following embodiments and aspects thereof are described and illustrated in conjunction with systems, tools and methods, which are meant to be exemplary and illustrative, not limiting in scope.

There is provided herein, according to an embodiment of the invention a reusable endoscopic water bottle assembly, the assembly comprising: a cap having internal threading, a port, and a first portion of a coupling system; a supply tube assembly, having a distal end and a proximal end, wherein the supply tube is configured to attach to the cap using a connector; a water bottle comprising an externally threaded neck configured to attach to said internal threading of the cap; and a second portion of said coupling system configured to attach to an endoscopic main control unit and configured to attach to the first portion of the coupling system.

In one embodiment, the externally threaded neck of said water bottle comprises a groove for receiving a sealing member, and wherein the sealing member is positioned between the water bottle and the cap.

In one embodiment, the cap further comprises a tube holder. Further, the tube holder is configured to receive and releasably hold the supply tube connector, wherein the supply tube connector has a plurality of protrusions along its circumference that fit into corresponding grooves formed within a receiving surface of the tube holder. Still further, the tube holder has an opening with a diameter that is approximately the same size as or slightly smaller than a diameter of the connector so that the connector is held securely in the tube holder using both friction and said tube holder grooves and said connector protrusions.

In one embodiment, the supply tube assembly is connected to the cap at its proximal end and using the connector and is connected to an air/water inlet of an endoscope at its distal end. Further, the supply tube assembly comprises a fitting at its proximal end for coupling the supply tube to the port located on the cap.

In one embodiment, the supply tube assembly further comprises an outer tube and an inner tube such that said inner tube extends into the water bottle.

In one embodiment, the supply tube assembly further comprises a CO₂ inlet port connected to a CO₂ insufflator.

In one embodiment, the first portion of the coupling system comprises a hanger plug integrally formed on the cap and said second portion of the coupling system comprises a hanger socket attached to the side of the endoscopic main control unit. Further, the hanger plug is adapted to slide into a groove located on the hanger socket. Still further, the hanger socket further comprises a wing portion configured to prevent the hanger plug from being disengaged by a horizontal pull force.

In another embodiment, the first portion of the coupling system comprises a first magnet affixed to the cap and the second portion of the coupling system comprises a second magnet affixed to a side of the main control unit, wherein said first magnet and said second magnet have opposite polarities, forming a magnetic coupling pair.

In one embodiment, the water bottle, cap and supply tube sub-assembly are autoclavable.

In another embodiment, the present specification describes a flushing adaptor for connecting a fluid source to a dual lumen supply tube of an endoscopic water bottle, the flushing adaptor comprising: a housing comprising a top end, a bottom end, and an outer body that extends from said top end and said bottom end; a first opening at the top end of the housing comprising a female luer component that is configured to receive a male luer component of the fluid source; a second opening at the bottom end of the housing adapted for receiving a connector of a supply tube, wherein said supply tube comprises an inner tube and an outer tube, with space therebetween, forming two concentric lumens; and a chamber within the housing for fluidly connecting said first and second openings and forming a passage therebetween, wherein said chamber is physically configured to enable fluid received from said first opening to be substantially uniformly distributed to each lumen in the supply tube through said connector.

In one embodiment, the outer body of the housing is of substantially cylindrical shape.

In one embodiment, the second opening is internally configured with a plurality of grooves to engage said connector of the supply tube.

In one embodiment, the chamber has a substantially cylindrical shape and receives at least one cleaning fluid by applying pressure to the fluid source when the fluid source is connected to the first opening. Further, the supply tube assembly comprises a CO₂ inlet port connected to a CO₂ insufflator for delivering air through the space between the two concentric lumens to pressurize an interior of the water bottle such that fluid flows through the inner lumen of the dual lumen supply tube at a desired rate.

In another embodiment, the present specification is directed towards a system for flush cleaning a dual lumen supply tube of an endoscopic water bottle, the system comprising: a connector at one end of the dual lumen supply tube; a fluid source with a male luer end; and a flushing adaptor, comprising: a housing comprising a top end, a bottom end, and an outer body that extends from said top end and said bottom end; a first opening at the top end of the housing comprising a female luer component that is configured to receive the male luer component of the fluid source; a second opening at the bottom end of the housing adapted for receiving the connector of a supply tube, wherein said supply tube comprises an inner tube and an outer tube, forming two concentric lumens; and a chamber within the housing for fluidly connecting said first and second openings and forming a passage therebetween, wherein said chamber is physically configured to enable fluid received from said first opening to be substantially uniformly distributed to each lumen in the supply tube through said connector.

The aforementioned and other embodiments of the present shall be described in greater depth in the drawings and detailed description provided below.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1 is an illustration of a reusable endoscopic water bottle assembly attached to an endoscopic main control unit;

FIG. 2a is an illustration of an embodiment of a reusable endoscopic water bottle assembly, detached from an endoscopic system;

FIG. 2b is an exploded view of the reusable endoscopic water bottle assembly of FIG. 2a;

FIG. 3 is an illustration of an embodiment of a water bottle that can be used with the reusable endoscopic water bottle assembly described in the present specification;

FIG. 4 is an illustration of one embodiment of a supply tube assembly that can be used with the reusable endoscopic water bottle assembly described in the present specification;

FIG. 5 is an illustration of one embodiment of a water bottle cap that can be used with the reusable endoscopic water bottle assembly described in the present specification;

FIG. 6a is an illustration showing the supply tube assembly of FIG. 4, held in a tube holder according to one embodiment;

FIG. 6b is an illustration showing the supply tube assembly of FIG. 4, held in a tube holder according to another embodiment;

FIG. 7a is an illustration depicting a front view of a hanger socket in accordance with an embodiment of the present invention;

FIG. 7b is an illustration depicting a back view of a hanger socket in accordance with an embodiment of the present invention;

FIG. 8 is a cross-sectional view through a longitudinal axis of the reusable endoscopic water bottle assembly of FIG. 2a;

FIG. 9a is a cross-sectional view of a flushing adaptor of the present invention;

FIG. 9b is a perspective view of the flushing adaptor shown in FIG. 9a;

FIG. 10a is an exploded view of a system for flush cleaning a dual lumen supply tube of a reusable endoscopic water bottle, such as the bottle shown in FIG. 2a, using the flushing adaptor; and

FIG. 10b is an illustration of an assembled view of the system shown in FIG. 10a.

### DETAILED DESCRIPTION

The present invention is directed towards advantageous placement/positioning of a reusable endoscopic water bottle assembly within the operating environment of an endoscopic system. Accordingly, in one embodiment the reusable endoscopic water bottle assembly is removably, yet securely, attached to an endoscopic main control unit using a coupling system.

The present invention is also directed towards a system for flush cleaning a dual lumen tube of the reusable endoscopic water bottle assembly using a flushing adaptor. The flushing adaptor is used to connect a fluid source to the dual lumen tube of the endoscopic water bottle assembly and incorporates a chamber or cavity that is geometrically shaped to enable cleaning fluid received from the fluid source to be uniformly distributed to both lumens of the dual lumen tube.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention. In the description and claims of the application, each of the words “comprise” “include” and “have”, and forms thereof, are not necessarily limited to members in a list with which the words may be associated.

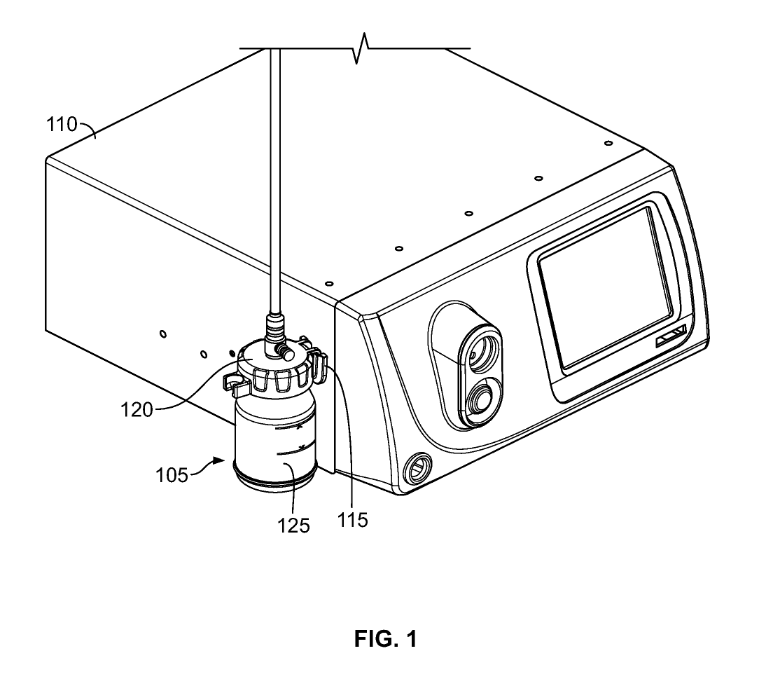

FIG. 1 depicts a reusable endoscopic water bottle assembly 105 that, in accordance with an aspect of the present invention, is removably, yet securely, attached to an endoscopic main control unit 110. Main control unit 110 controls an endoscope and typically comprises at least a video system with a display. The reusable water bottle assembly 105 is typically attached to a side of the main control unit 110 using a coupling system 115. The attachment of the reusable water bottle assembly 105 to the Main Control Unit 110 offers an advantageous placement of the bottle such that it is easy to manipulate, is securely held using a coupling system, and does not hinder or obstruct the endoscopic operational environment while being securely held.

In accordance with an embodiment of the present invention, the coupling system 115 comprises a hanger plug and socket pair. In one embodiment, the hanger plug is integrally formed on a cap 120 of the water bottle 125 while the hanger socket is removably, yet securely, affixed to the main control unit 110 and is configured to receive the hanger plug.

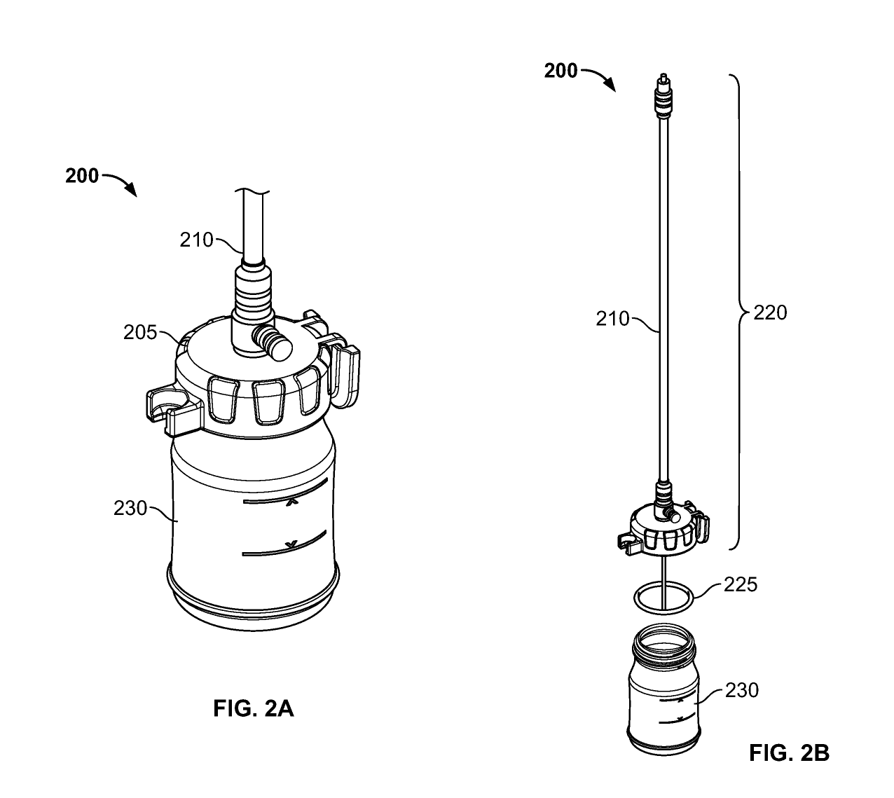

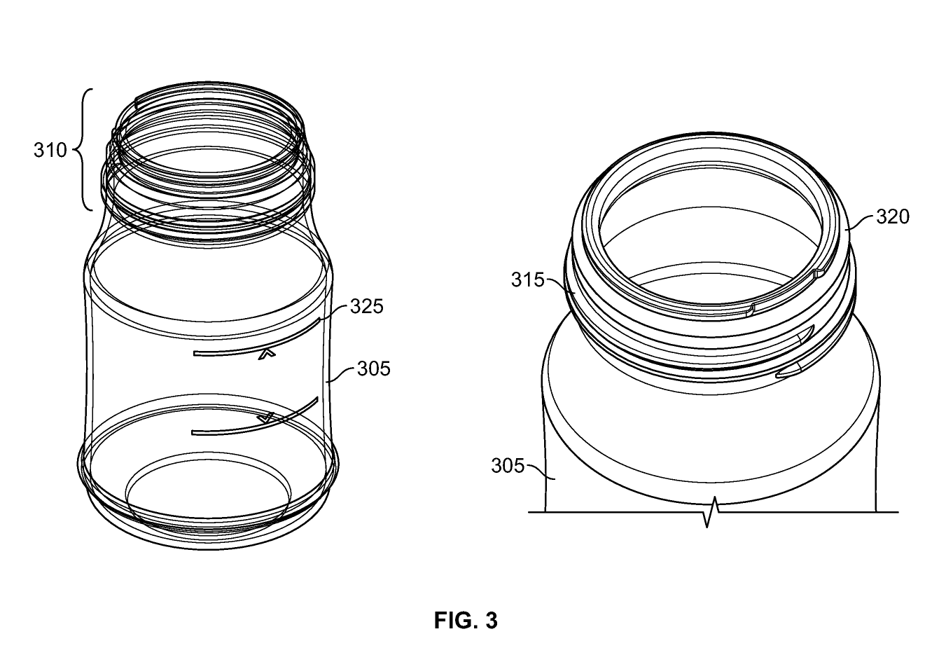

FIGS. 2a and 2b show assembled and exploded views, respectively, of a reusable endoscopic water bottle assembly 200. In one embodiment, the water bottle assembly 200 comprises three sub-assemblies or components including a cap and supply tube sub-assembly 220, a sealing member 225, such as an O-ring or a gasket, and water bottle 230. During use, the cap and supply tube sub-assembly 220 is secured onto the water bottle 230 and sealed thereto, using the sealing member 225. In one embodiment, the cap portion of the assembly 220 contains internal threading that can be used to secure the cap to the water bottle which has an externally threaded neck, described below with respect to FIG. 3. A tube 210 runs through the cap and supply tube sub-assembly 220 to supply fluid (water and/or gas) to an endoscope.

Persons of ordinary skill in the art would appreciate that for endoscopic procedures, a flow of sterile water, from the water bottle 230, is provided across the optical assemblies of a tip of the endoscope to prevent buildup of materials (for example, surgical debris and body fluids such as, blood, feces and digestive fluid) on the tip. In one embodiment, this flow of water operates like a windshield wiper/washer assembly. After use, in one embodiment, the assembly 200 is disassembled into its three sub-assemblies/components for sterilization, such as by glutaraldehyde disinfection and/or autoclaving, and, therefore, reuse. In another embodiment, the assembly is disposable.

In one embodiment the cap 205, tube 210 and water bottle 230 are made from a thermoplastic elastomer (TPE) and/or a thermoset elastomer that have sufficient pliability and/or are capable of frictionally engaging with other structures (for example, endoscope ports) to provide a substantially air tight seal and at the same time allow a user to easily install or disassemble the components. Such pliability and frictional engaging capability may be achieved by over-molding or affixing a soft elastomer onto at least portions of hard-plastic sub-assemblies or components. Such a process provides a suitable seal without requiring ultrasonic welding of small components or the designing of complex coupling structures.

As shown in FIG. 3, the water bottle 305 may be of any suitable size and/or type of water bottle. The water bottle 305 may be, for example, a one liter water bottle of a conventional type used in hospitals. In one embodiment, the water bottle 305 is extrusion blow-molded and has a total volume of 340 milliliters which, when filled to line mark 325 has a total volumetric capacity of 210 milliliters. In one embodiment, the bottle 305 is auto-clavable, made from a biocompatible material and filled with sterile water. Sterile water is used since the water will pass through the interior of the human body during the process of cleaning the optical assemblies positioned on the tip of the endoscopic instrument. The water bottle 305 also has an externally threaded neck 310. In one embodiment, the externally threaded neck 310 comprises trapezoidal threads 315 enabling 1.5 free rotation turns and a 0.5 turn for achieving a seal. The externally threaded neck 310 comprises a groove or recess for receiving a sealing member, such as an O-ring 320 to ensure a suitable seal when a cap (not shown) is secured, using said threads, to the water bottle 305, preventing leakage or dispensing of the fluid from the interior of the bottle during transportation and storage of the water bottle 305.

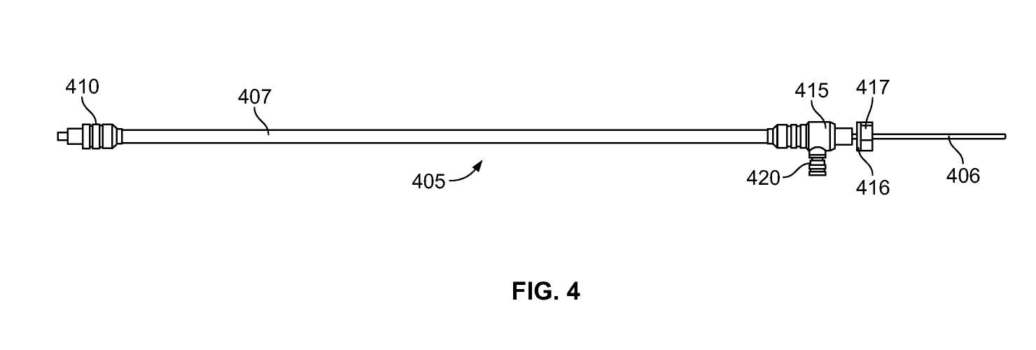

FIG. 4 is an illustration of one embodiment of a supply tube assembly that can be used with the reusable endoscopic water bottle assembly described in the present specification. Referring now to FIG. 4, a supply tube assembly 405 has a connector 410 at a distal end to connect the supply tube assembly 405 to an air/water inlet of an endoscope. A fitting 415 at the proximal end includes a silicone washer 416 and a nut 417 to enable the supply tube assembly 405 to be securely coupled to the cap of the water bottle, as shown in FIG. 2a. The supply tube assembly 405 further includes an inner tube 406 and an outer tube 407, forming a dual lumen tube. The inner tube 406 extends into the water bottle resting at or near the bottom of the water bottle in order to draw fluid from the water bottle. The outer tube 407 is connected to the cap of the water bottle, forming the cap and supply tube sub-assembly 220 of FIG. 2b.

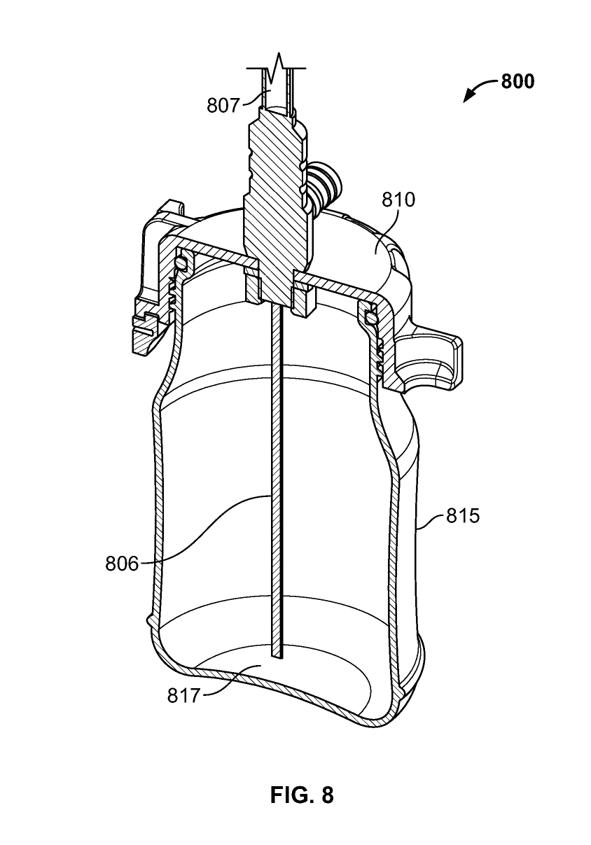

FIG. 8 is a cross-sectional view through a longitudinal axis of the reusable endoscopic water bottle assembly of FIG. 2a. Referring now to FIG. 8, water bottle assembly 800 comprises an outer tube 807 connected to the cap 810 of water bottle 815 and an inner tube 806 that extends through the cap 810 and into the bottle 815 to rest at or near the bottom 817. It should be appreciated to those of ordinary skill in the art that inner tube 806 is positioned such that it does not obstruct the flow of fluid.

During use, air is delivered through the area between the inner tube and the outer tube in order to pressurize the interior of the water bottle. This enables fluid to flow through the inner tube 806 and into the endoscope at a desired rate. Referring back to FIG. 4, in accordance with an embodiment, the supply tube assembly 405 also comprises a carbon dioxide (CO₂) inlet port 420 to provide CO₂ access through tubing. In one embodiment, the inlet port 420 comprises a one way valve with luer lock for connection to a CO₂ source, such as a CO₂ insufflator. It has been found that the use of carbon dioxide (CO₂) insufflation, compared to the use of pressured air, can improve post-procedure patient comfort since CO₂ is more easily absorbed by the body. For example, use of CO₂ may be useful for long endoscopic exams, such as endoscopic retrograde cholangiopancreatogram (ERCP), enteroscopy, and colonoscopy.

Luer connection systems typically are associated with the interconnection of syringes, catheters, hubbed needles, IV tubes, and the like. A luer connection system consists of round male and female interlocking tubes that may be slightly tapered to hold together with a pressure/twist fit. In use, a male luer connector component slips into the female luer connector component to form a secure connection. Persons of ordinary skill in the art should appreciate that in alternative embodiments non-luer connection systems may be used such as, for example, disclosed in PCT Application No. PCT/FR00/003349 01039827 entitled “Non-luer lock connection of a male connector with a female connector for medical devices”. Another example is that of the Portex® CorrectInject™ Safety System from Smiths Medical comprising a series of interlocking connections featuring non-luer tapers to reduce the risk of misconnection and the potential for medication administration errors.

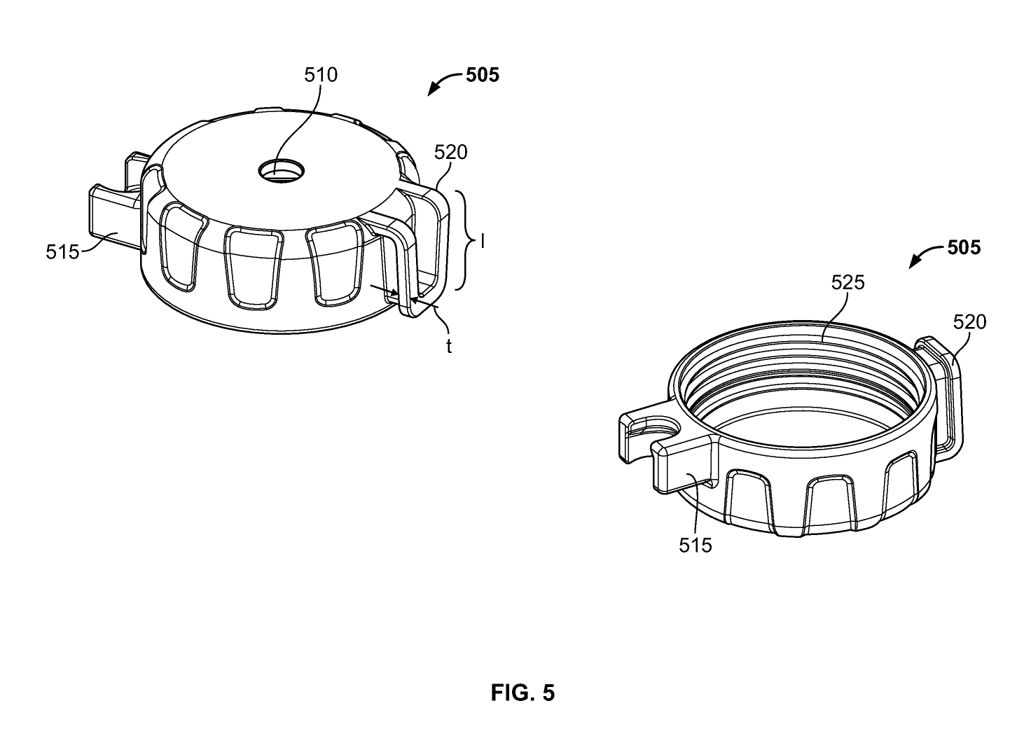

As shown in FIG. 5, the cap 505 described in the present specification comprises a port 510 to receive the supply tube assembly 405 of FIG. 4. In accordance with an embodiment, the cap 505 also comprises a hanger plug 520 and an optional tube holder 515 both of which are integrally formed on the circumference of the cap 505. In one embodiment, “integrally formed” means that the tube holder and hanger plug are injection molded along with and as integral parts of the cap 505. In an alternate embodiment, the cap 505 comprises only the hanger plug 520. In another embodiment, a hanger plug and/or tube holder are welded, glued, or attached in any other manner to the cap 505. In one embodiment, the cap 505 comprises one or more threads 525 on an interior surface of the cap 505 for removably, yet securely attaching the cap 505 to the water bottle, as shown in FIG. 2a. In one embodiment, interior threads 525 of the cap may be sized to fit over various shapes and sizes of exterior threads that may exist on the neck of the water bottle. In another embodiment, 525 may be designed to specifically mate with one type of water bottle thread type, such as those for water bottle 305 shown in FIG. 3.

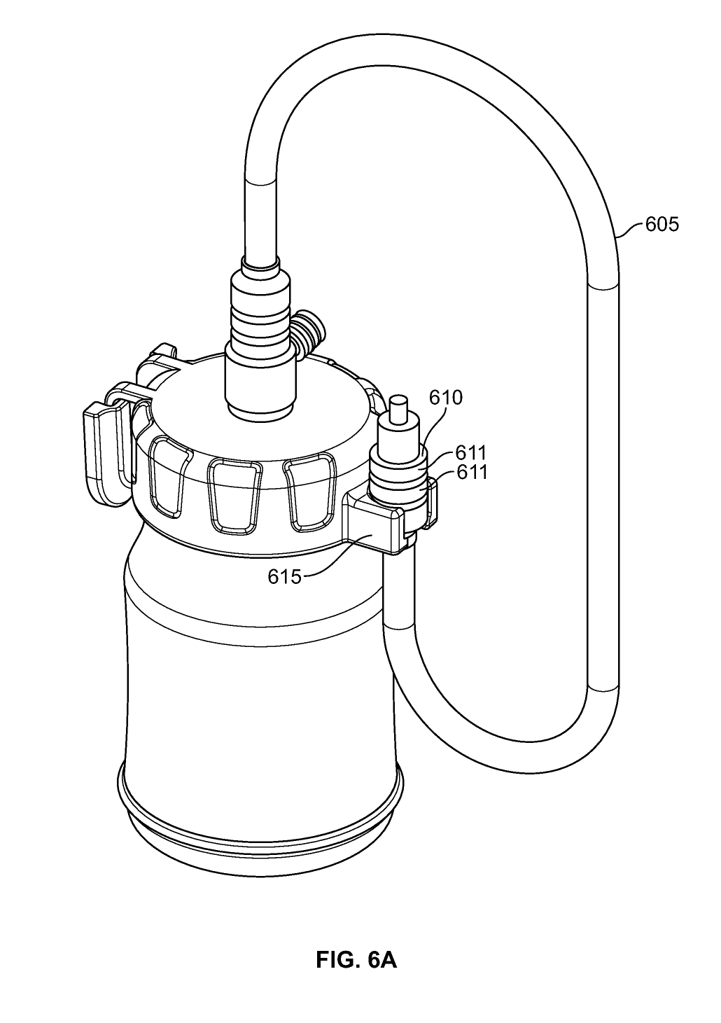

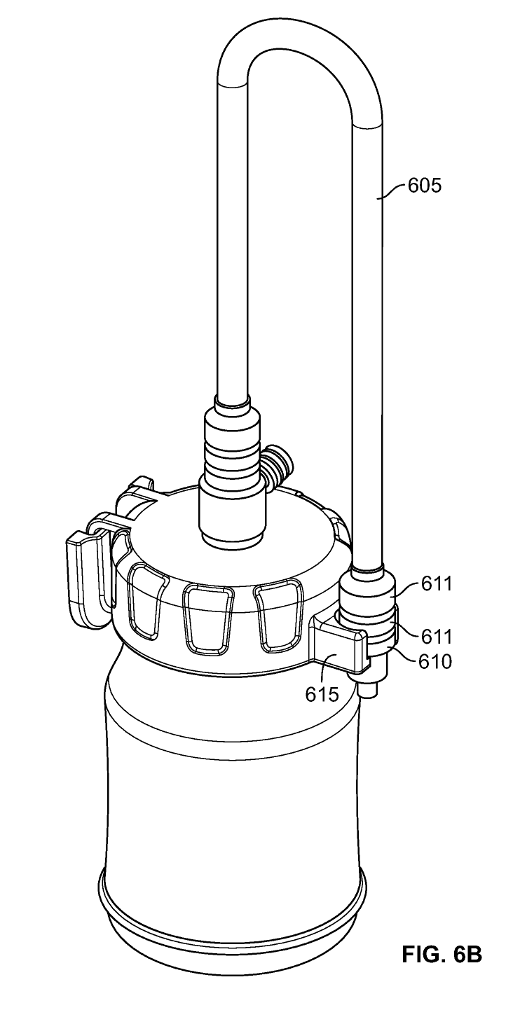

The optional tube holder 515 allows the supply tube assembly 405 of FIG. 4 to be advantageously and removably anchored into the optional tube holder 515. FIG. 6a is an illustration showing the supply tube assembly of FIG. 4, held in a tube holder according to one embodiment. FIG. 6b is an illustration showing the supply tube assembly of FIG. 4, held in a tube holder according to another embodiment. Now referring to FIG. 6a, supply tube 605 is releasably held in the tube holder 615 such that the supply tube 605 is looped in from below and connector 610 is directed vertically upwards. FIG. 6b shows another embodiment of the supply tube 605 being releasably held in the tube holder 615 such that the tube 605 is looped in and coupled from above so that the connector 610 is directed vertically downwards. In one embodiment, the connector 610 has a plurality of protrusions 611 along its circumference that snug-fit into corresponding grooves formed within the receiving surface of the optional tube holder 615. In one embodiment, the connector 610 is pushed into or snapped into the tube holder 615 opening which is of a slightly smaller diameter than the diameter of the connector 610 such that the connector 610 is held securely in the holder 615 with the help of the grooves/protrusions 611 and friction produced by a tight-fit.

Persons of ordinary skill in the art should appreciate that while some embodiments of the tube holder 615 have been described above, alternate embodiments of tube holder mechanisms may be employed to enable releasable attachment of the connector/tube. For example, the holding mechanism could comprise clips, snaps, hooks, clasps, a female/male attachment pair, or any other attachment component. Similarly, while the orientation of the holder in one embodiment is vertical such that the connector is held in a vertical position, in alternate embodiments the holder is oriented horizontally such that the connector is held in a horizontal position. In still alternate embodiments, the holder can be connected in any orientation such that the tubing does not interfere with operation of the water bottle assembly or the endoscopic assembly.

Referring back to FIG. 5, hanger plug 520 enables the capped water bottle to be attached typically to a side of the endoscopic main control unit as shown in FIG. 1. For attachment, a corresponding hanger socket is attached to the side of the main control unit.

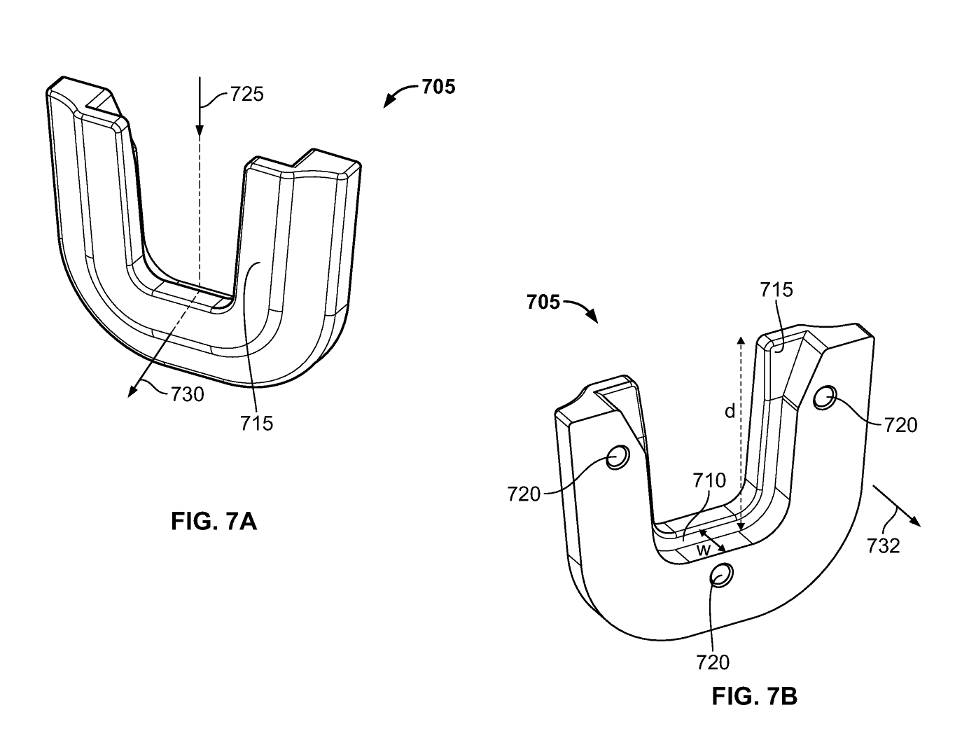

FIG. 7a is an illustration depicting a front view of a hanger socket in accordance with an embodiment of the present invention, while FIG. 7b is an illustration depicting a back view of a hanger socket in accordance with an embodiment of the present invention. Referring to FIGS. 7a and 7b simultaneously, hanger socket 705 comprises a U-shaped groove portion 710 having a depth ‘d’ that is capable of receiving at least a portion of the hanger plug 520 having a total length “l” so that when the hanger plug is slid in a vertically downward direction 725, groove 710 of the hanger socket receives at least a portion of the hanger plug in a snug, secure, yet removable fashion. Further, the thickness ‘t’ of the hanger plug 520 of FIG. 5 is slightly smaller than the width “w” of groove portion 710 so that when the hanger plug is slid in a vertically downward direction 725, the groove portion of the hanger socket receives the hanger plug in a snug, secure, yet removable fashion.

In one embodiment, a wing portion 715 acts as a stop on the front side and prevents the plug from disengaging from the socket in a forward direction as a result of any horizontal pull force 730. The hanger socket 705 comprises a plurality, such as three, of threaded holes 720 to enable the socket to be firmly screwed to a side of the main control unit. The main control unit acts a stop on the backside to prevent the plug from disengaging from the socket in a backward direction as a result of any horizontal pull force 732.

Referring back to FIG. 1, while the coupling system 115, in one embodiment, comprises a hanger plug and socket pair (as described above with reference to FIGS. 5 and 7), alternate embodiments may comprise other connection systems that are easily connected/disconnected but securely fixed. For example, the connection system may include a magnetic coupling pair where a first magnet is fixed to the cap of the water bottle and a second magnet, having polarity opposite to the first, is affixed to a side of the main control unit. Bringing the first magnet close to the second would result into a strong magnetic coupling to enable the capped water bottle to be removably, yet securely, attached to the main control unit.

Additional examples may include clips, snaps, clasps, hooks, Velcro, a female/male attachment pair, and other connection systems that enable removable, yet secure, coupling as would be advantageously evident to persons of ordinary skill in the art. For example a clip assembly is disclosed in U.S. Pat. No. 6,457,691 entitled “Bicycle water bottle clip assembly” while another clip assembly is disclosed in U.S. Pat. No. 5,839,632 titled “Rotatable water bottle holder”. A bayonet snap assembly is disclosed in U.S. Pat. No. 4,830,240 entitled “Water bottle cage and method” while a clasping assembly is disclosed in U.S. Pat. No. 6,837,472 entitled “Releasable bottle holder”.

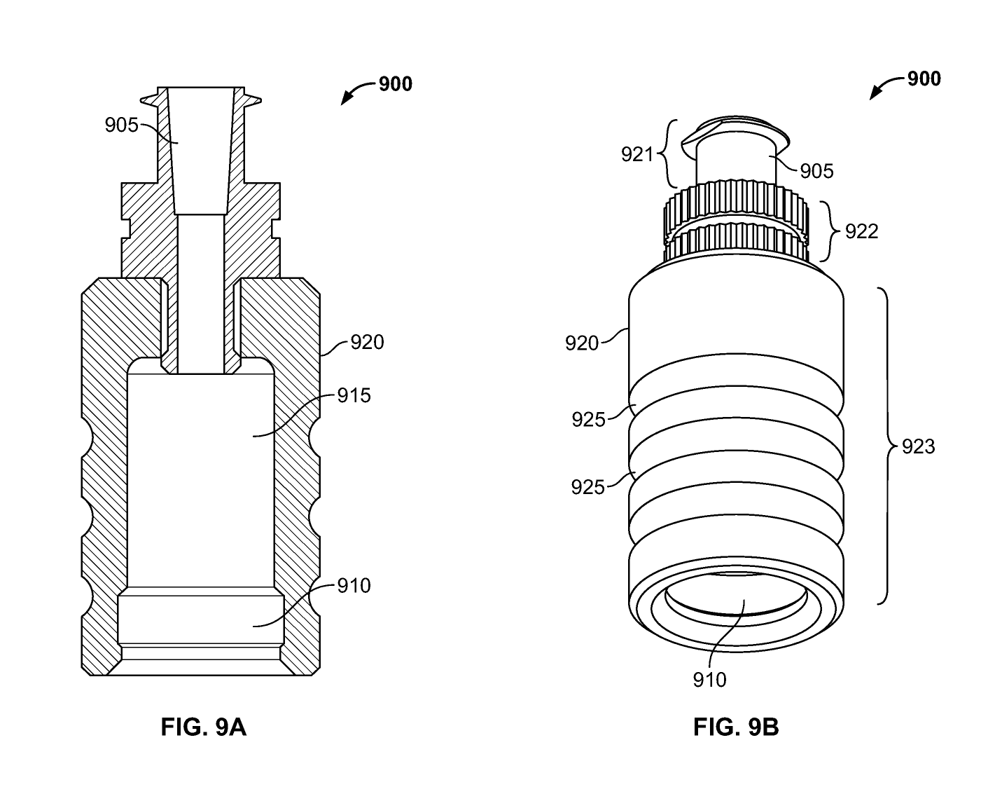

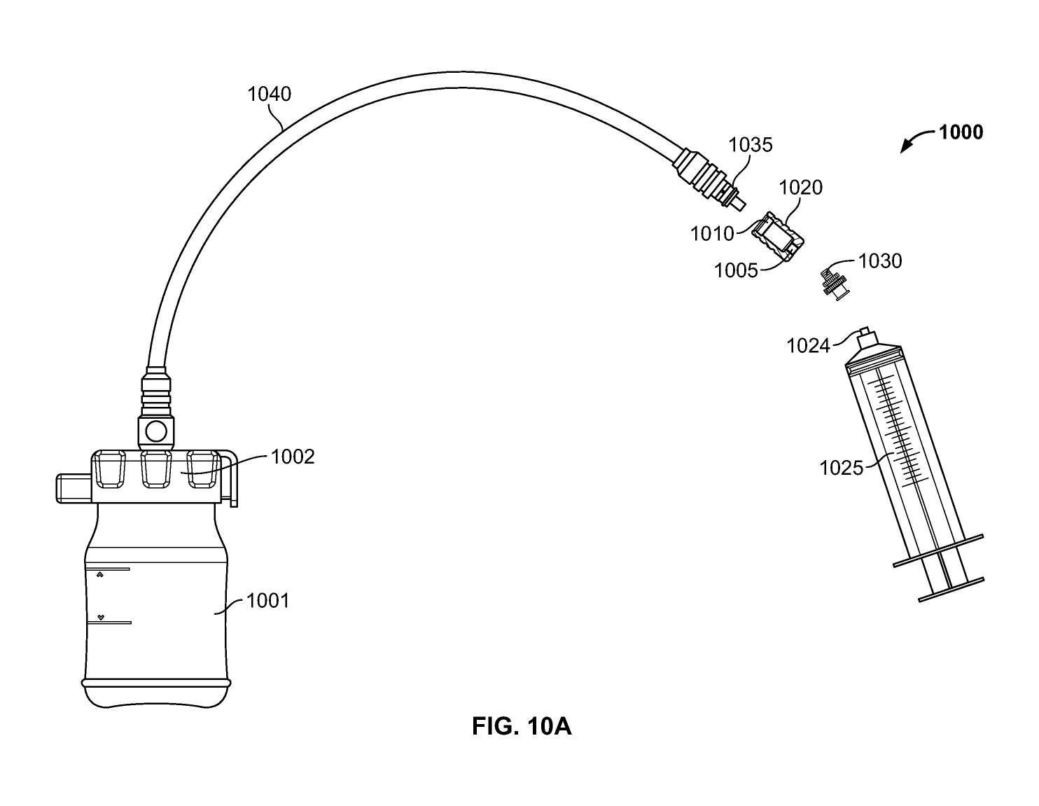

FIGS. 9a and 9b are cross-sectional and perspective views, respectively, of a flushing adaptor 900 in accordance with an embodiment of the present invention. In one embodiment, flushing adaptor 900 comprises an outer housing or casing body 920 having a first receptacle/opening 905 at a first, top end, a second receptacle/opening 910 at a second, bottom end. In an embodiment, housing body 920 is substantially cylindrical. A hollow distribution chamber or cavity 915 is positioned within said outer housing that fluidically connects first opening 905 and second opening 910, forming a fluid passage between openings 905 and 910. In one embodiment, outer surfaces of portions 921, 922 of the housing 920, covering the outer surfaces of receptacle/opening 905, are also substantially cylindrical. In one embodiment, portions 921, 922 have a smaller diameter than the diameter of portion 923 which contains chamber 915 and receptacle/opening 910. However, in alternate embodiments the outer surfaces of portions 921, 922 and 923 may be of the same cylindrical diameter. Persons of ordinary skill in the art should appreciate that the housing 920 in alternate embodiments may have a substantially square or rectangular shape on the outside.

In one embodiment, receptacle/opening 905 is appropriately dimensioned and internally configured/shaped as a female luer for connecting, mating with or receiving a male luer component of a fluid source. The fluid source is used to infuse cleaning fluid through the flushing adaptor 900 and in one embodiment comprises a syringe with a male luer connector end. A generic adaptor is used to connect the male luer end/component of the syringe and the receptacle/opening 905. The cleaning fluid, in one embodiment, comprises liquids such as water, solutions such as saline water or any other liquid that offers washing, disinfecting and sanitization function. Referring now to FIGS. 2a, 2b, 4, 9a and 9b simultaneously, in one embodiment, the receptacle/opening 910 is appropriately dimensioned and internally configured, shaped or contoured with a plurality of grooves to receive and hold the connector 410 at the distal end of the dual lumen supply tube assembly 405 of the reusable endoscopic water bottle 230. The proximal end of the dual lumen supply tube assembly is securely coupled to the cap of the water bottle, as shown in FIG. 2a. The dual lumen supply tube assembly 405 comprises coaxial inner and outer lumens of two concentric tubes, one within the other.

Referring back to FIGS. 9a and 9b, the chamber or cavity 915 serves as reservoir to receive the cleaning fluid, from the fluid source such as a syringe, through the receptacle/opening 905, and distribute the cleaning fluid to both inner and outer lumens of the dual lumen supply tube, through the receptacle/opening 910. The chamber or cavity 915 is provided with an internal geometric shape that enables the cleaning fluid to be dispensed in substantially equal, even or uniform and streamlined flow to both the inner and outer lumens of the dual lumen supply tube. In one embodiment, the internal geometric shape of the chamber or cavity 915 is substantially cylindrical. In one embodiment, the outer surface of the housing 920 incorporates a plurality of contours or grooves 925 to enable a good grip during handling of the flushing adaptor 900.

The flushing adaptor is made from a durable, long-lasting material such as stainless steel, medical grade thermo-plastic polymer/elastomer or any other suitable material known in the art that provides robustness to the flushing adaptor and is also capable of withstanding the same chemical environment as the endoscopic bottle and/or dual lumen supply tube while cleaning.

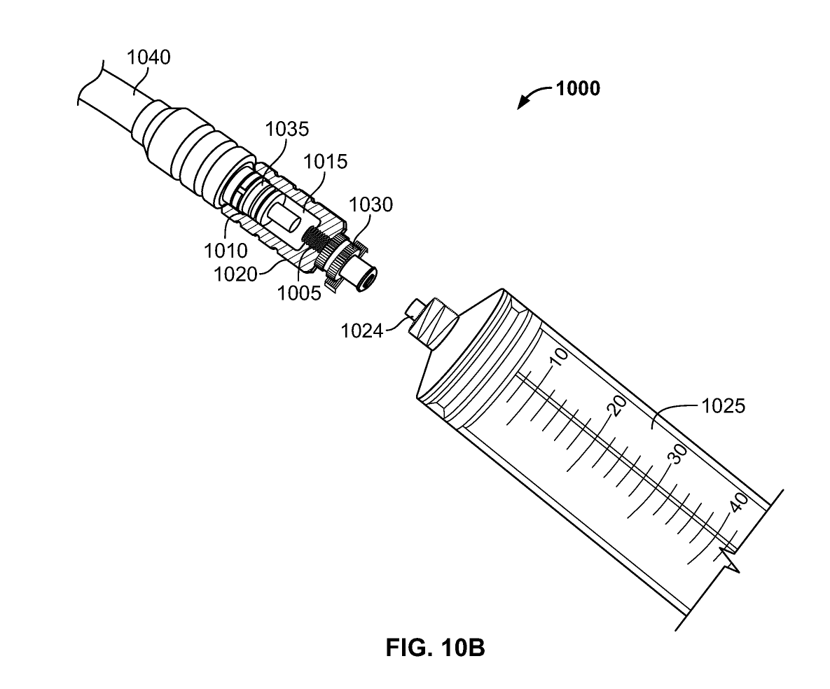

FIG. 10a illustrates, in accordance with an embodiment, an exploded view of a system 1000 for flush cleaning a dual lumen supply tube 1040 of a reusable endoscopic water bottle 1001 (such as for example, the dual lumen supply tube 210 of the reusable endoscopic water bottle 230 of FIGS. 2a and 2b), using flushing adaptor 1020 described in the present specification. Flushing adaptor 1020 is shown with a receptacle/opening 1005, at one end, internally configured as a female luer for receiving a male luer end/component 1024 of a fluid source 1025, such as a syringe. A generic fluid source adaptor 1030 is used to connect between the male luer end 1024 of the syringe 1025 and the receptacle/opening 1005 of the flushing adaptor 1020. At the other end of flushing adaptor 1020, a receptacle/opening 1010, having internal grooves, is used to receive and hold a metal connector 1035 of the dual lumen supply tube 1040 of the reusable endoscopic water bottle 1001. The tube 1040 comprises coaxial double lumen of two concentric inner and outer tubes. The concentric tubes merge at its two ends such that the tube 1040 connects to a bottle cap 1002 at its proximal end and connects to the metal connector 1035 at its distal end. The metal connector 1035 has separate channels to maintain a separation between the two lumens.

FIG. 10b illustrates an assembled view of the system 1000 wherein the metal connector 1035 of the dual lumen supply tube 1040, located at the distal end of the supply tube, is held within the receptacle/opening 1010 (of the flushing adaptor 1020) while the receptacle/opening 1005 holds the generic adaptor 1030 which in turn connects to the male luer end 1024 of the syringe 1025. During flushing/cleaning operation, the hollow chamber or cavity 1015 receives cleaning fluid, such as water, saline or any other cleansing liquid contained in the syringe 1025, when requisite pressure is applied to a plunger of the syringe 1025 to force-in cleaning fluid through opening/receptacle 1005. The cleaning fluid received within the chamber or cavity 1015 is distributed evenly or uniformly to the lumens of the dual lumen supply tube 1040 through the metal connector 1035 thereby cleaning the dual lumen supply tube 1040 either when connected to or disconnected from endoscopic water bottle 1001.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A reusable endoscopic water bottle assembly, the assembly comprising:
a cap having internal threading, a port, and a first portion of a coupling system;
a supply tube assembly, having a distal end and a proximal end, wherein the supply tube is configured to attach to the cap using a connector;
a water bottle comprising an externally threaded neck configured to attach to said internal threading of the cap; and
a second portion of said coupling system configured to attach to an endoscopic main control unit and configured to attach to the first portion of the coupling system.

2. The assembly according to claim 1, wherein the externally threaded neck of said water bottle comprises a groove for receiving a sealing member, and wherein the sealing member is positioned between the water bottle and the cap.

3. The assembly according to claim 1, wherein the cap further comprises a tube holder.

4. The assembly according to claim 3 wherein the tube holder is configured to receive and releasably hold said connector, wherein the connector has a plurality of protrusions along its circumference that fit into corresponding grooves formed within a receiving surface of the tube holder.

5. The assembly according to claim 4 wherein the tube holder has an opening with a diameter that is approximately equal to a diameter of the connector so that the connector is held securely in the tube holder using both friction and said tube holder grooves in physical communication with said connector protrusions.

6. The assembly of claim 1, wherein the supply tube assembly is connected to the cap at its proximal end and using the connector and is connected to an air/water inlet of an endoscope at its distal end.

7. The assembly of claim 4, wherein the supply tube assembly further comprises a fitting at its proximal end for coupling the supply tube to the port located on the cap.

8. The assembly according to claim 1, wherein the supply tube assembly further comprises an outer tube and an inner tube such that said inner tube extends into the water bottle.

9. The assembly according to claim 1, wherein the supply tube assembly further comprises a CO₂ inlet port connected to a CO₂ insufflator.

10. The assembly according to claim 1, wherein said first portion of the coupling system comprises a hanger plug integrally formed on the cap and said second portion of the coupling system comprises a hanger socket attached to the side of the endoscopic main control unit.

11. The assembly according to claim 9, wherein the hanger plug is adapted to slide into a groove located on the hanger socket.

12. The assembly according to claim 10, wherein the hanger socket further comprises a wing portion configured to prevent the hanger plug from being disengaged by a horizontal pull force.

13. The assembly according to claim 1, wherein said first portion of the coupling system comprises a first magnet affixed to the cap and said second portion of the coupling system comprises a second magnet affixed to a side of the main control unit, wherein said first magnet and said second magnet have opposite polarities, forming a magnetic coupling pair.

14. The assembly according to claim 1, wherein the water bottle, cap and supply tube sub-assembly are autoclavable.

15. A flushing adaptor for connecting a fluid source to a dual lumen supply tube of an endoscopic water bottle, the flushing adaptor comprising:
a housing comprising a top end, a bottom end, and an outer body that extends from said top end and said bottom end;
a first opening at the top end of the housing comprising a female luer component that is configured to receive a male luer component of the fluid source;
a second opening at the bottom end of the housing adapted for receiving a connector of a supply tube, wherein said supply tube comprises an inner tube and an outer tube, with space therebetween, forming two concentric lumens; and
a chamber within the housing for fluidly connecting said first and second openings and forming a passage therebetween, wherein said chamber is physically configured to enable fluid received from said first opening to be substantially uniformly distributed to each lumen in the supply tube through said connector.

16. The flushing adaptor of claim 15, wherein the outer body said housing is of substantially cylindrical shape.

17. The flushing adaptor of claim 15, wherein said second opening is internally configured with a plurality of grooves to engage said connector of the supply tube.

18. The flushing adaptor of claim 15 wherein the chamber has a substantially cylindrical shape.

19. The flushing adaptor of claim 15, wherein the chamber receives at least one cleaning fluid by applying pressure to the fluid source when the fluid source is connected to the first opening.

20. The flushing adaptor of claim 19 wherein the supply tube assembly further comprises a CO₂ inlet port connected to a CO₂ insufflator for delivering air through the space between the two concentric lumens to pressurize an interior of the water bottle such that fluid flows through the inner lumen of the dual lumen supply tube at a desired rate.

21. A system for flush cleaning a dual lumen supply tube of an endoscopic water bottle, the system comprising:
a connector at one end of the dual lumen supply tube;
a fluid source with a male luer end; and
a flushing adaptor, comprising:
a housing comprising a top end, a bottom end, and an outer body that extends from said top end and said bottom end;
a first opening at the top end of the housing comprising a female luer component that is configured to receive the male luer component of the fluid source;
a second opening at the bottom end of the housing adapted for receiving the connector of a supply tube, wherein said supply tube comprises an inner tube and an outer tube, forming two concentric lumens; and
a chamber within the housing for fluidly connecting said first and second openings and forming a passage therebetween, wherein said chamber is physically configured to enable fluid received from said first opening to be substantially uniformly distributed to each of the two concentric lumens in the supply tube through said connector.
